(19) United States
(12) Patent Application Publication
(10) Pub. No.: US 2005/0143794 A1
(43) Pub. Date: Jun. 30, 2005

(54) Sap adjuster

(76) Inventor: Hsiao Fu Yuan, Chang Hua (TW)

Correspondence Address: HSIAO FU YUAN, P.O. BOX 23-90, Chang-Hua City, TW

(21) Appl. No.: 10/747,501

(22) Filed: Dec. 30, 2003

(51) Int. Cl. A61F 7/00; A61F 7/12

(52) U.S. Cl. 607/96

(57) ABSTRACT

A sap adjuster comprises a plurality of hot compress units and a control box. Each hot compress unit has a heat-tolerable insulting rubber sheet and two electric heated sheets. The heat-tolerable insulting rubber sheet and the two electric heated sheets are combined by silicon. A short conductive wire serves to connect the two electric heated sheets. Each electric heated sheet is connected with a long conductive wire. One end surface of the control box is installed with a receptacle, two stud-like security seats and a power plug. Another end surface of the control box is installed with a temperature controller, a power switch, and a four hole receptacle.

### FIELD OF THE INVENTION

The present invention relates to sap adjusters, and particular to a sap adjuster which includes a plurality of hot compress units arranged in parallel and the plurality of hot compress units are enclosed by two large area heat-tolerable insulting rubber sheets so as to have a larger hot compress area.

### BACKGROUND OF THE INVENTION

The prior art health machine frequently uses electromagnetic waves or electric pulses as a means for treat. However, impulses are easily generated and the stability is thus low. Thereby, these kinds of prior arts are not so suitable. The inventor of the present invention ever designs a hot compress treating machine which includes a hot compress head, a temperature sensor, and a control box. The hot compress head is a rectangular insulator body. A front face thereof has a shallow and a deep recesses. Two sides of the recesses are installed the two electric heated sheets. Each electric heated sheet has two threaded holes. A short conductive wire serves to connect the two electric heated sheets. The power wires pass through the screw holes of the electric heated sheets to be connected to the outer terminals of the two electric heated sheets. The temperature sensors are placed in a long recess of an insulating body. The power wires of the temperature sensors and the electric heated sheets and the shielding conductive wire at one end of the temperature sensor pass through the threaded holes of the insulating body. A bottom plate is a rectangular plate. An upper side thereof has four threaded holes. Four screws serve to fix the bottom plate to the shallow recess of the insulating body. One threaded hole of the insulating body is installed with a joint. Another end thereof is engaged with a soft tube. The power wire, i.e., a shield conductive wire passes through the soft tube to a backside of the control box for connecting the indicators and temperature setting device. Furthermore, a cloth cover encloses the hot compress head. The backside of the control box is further connected to a power plug and a soft conducive tube and a front side thereof is installed with a power switch, a temperature controller, a current meter, and an indicator. The temperature controller includes a temperature setting device and a number display. A hold type fuse is connected between the power switch and a temperature setting device. The temperature setting device is further connected to a temperature controlling relay, and then is connected to the current meter and the indicator. The indicator is connected to the electric heated sheets of the hot compress head. The temperature setting device is connected to an overheat protecting circuit. The overheat protecting circuit is connected to an abnormal alarm device.

However, the prior art still has some defects which will be described here.

Firstly, the hot compress head is a rectangular insulating body. Four threaded holes are used in connection. Thereby, the connection is a hard-connection. When it is necessary to hot compress the back of the patient, the user will feel uneasy. Thereby, the curved portion of the body cannot suit the structure of the hot compress head. Thereby, the treating effect is not preferred.

Secondary, as shown in FIG. 1, the prior art hot compress way for a larger hot compress area is illustrated. When a larger area is necessary to hot compress, a plurality of hot compress heads 6 are assembled for achieving a desired effect. Thereby, some bandages 4 are needed to fix the hot compress heads. However, the fixing way is unstable. If the body of the patient moves, the user will feel inconvenient.

Thirdly, when more and more hot compress heads 6 are used, the power wires will wind complicatedly. If it is desired to separate the wires in force, the power wires will destroy so that power will lose.

Fourthly, a handle is added to the hot compress head for being held by the user, but the user is easy to hurt due t the heat from the hot compress sheets. Thereby, this prior art device is easy to use to hot compress a small area.

### SUMMARY OF THE INVENTION

Accordingly, the primary object of the present invention is to provide a sap adjuster comprises a plurality of hot compress units and a control box. Each hot compress unit has a heat-tolerable insulting rubber sheet and two electric heated sheets. The heat-tolerable insulting rubber sheet and the two electric heated sheet are combined by silicon. A short conductive wire serves to connect the two electric heated sheets. Each electric heated sheet being connected with a long conductive wire. Thereby, the sap adjuster is suitable to the curvature of human body. The sap adjuster of the present invention can be used with a cushion, pillow or mattress. Thereby, the use of the sap adjuster is not confined by positions and timings.

Another object of the present invention is to provide a sap adjuster, wherein a plurality of hot compress units are connected in parallel so as to be formed as a large area heat-tolerable insulting rubber sheet. The long conductive wires of electric heated sheets are collected at the edges of the large area heat-tolerable insulting rubber sheet. The upper sides and lower sides of the plurality of hot compress units are enclosed by two large area heat-tolerable insulting rubber sheets so that the whole sap adjuster of the present invention can be bent with the shape of the human body. Thus, the present invention is a device of matching ergonomics.

Moreover, in the present invention the power wires are arranged in order so that the power wires are not wound complicatedly.

The various objects and advantages of the present invention will be more readily understood from the following detailed description when read in conjunction with the appended drawing.

### BRIEF DESCRIPTION OF THE DRAWINGS

FIG. 1 shows the retaining way of the prior art holdable hot compress head.

FIG. 2 is a schematic view showing the structure of the present invention.

FIG. 3 is a structural exploded view of the sap adjuster of the present invention.

FIG. 4 is an assembled schematic view of the sap adjuster of the present invention.

FIG. 5 is assembled cross section view of the sap adjuster of the present invention.

FIG. 6 is a schematic view showing the front surface of the control box of the present invention.

FIG. 7 is a schematic view about the structure of the present invention.

FIG. 8 is a schematic view showing the structure of the present invention.

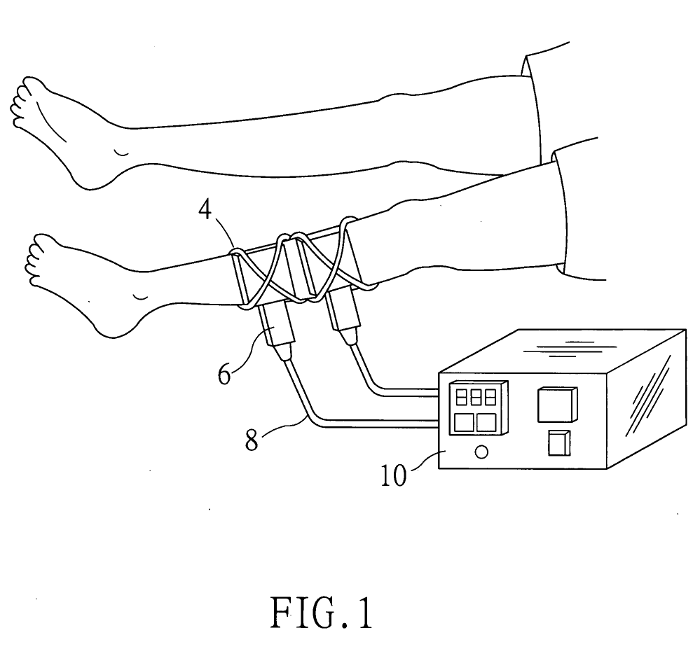

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

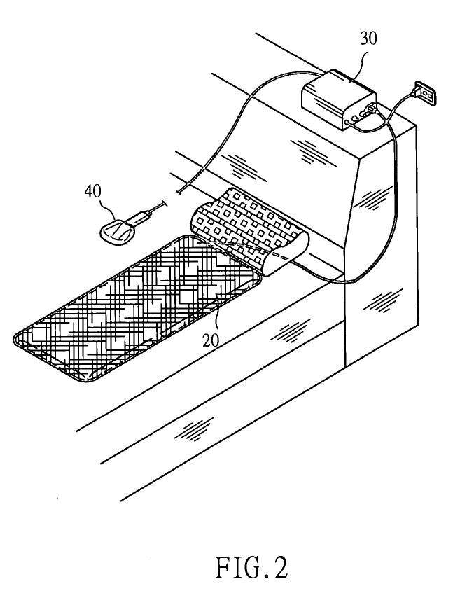

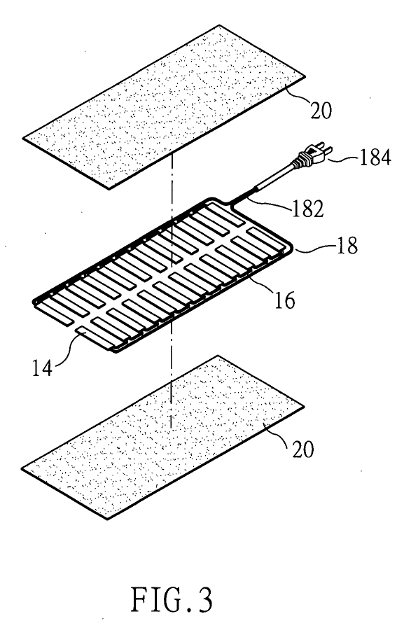

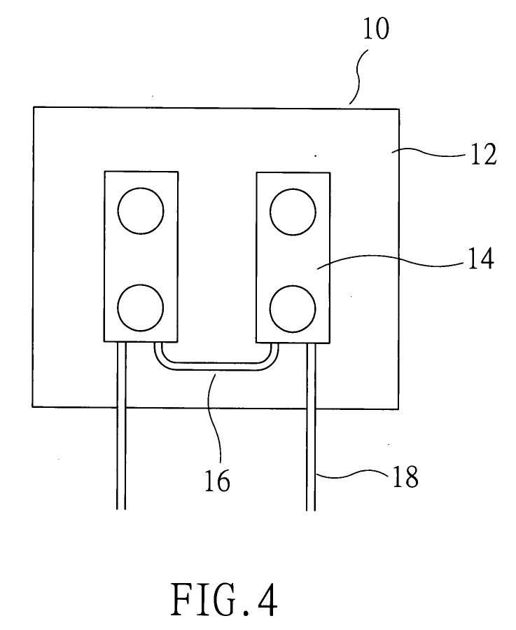

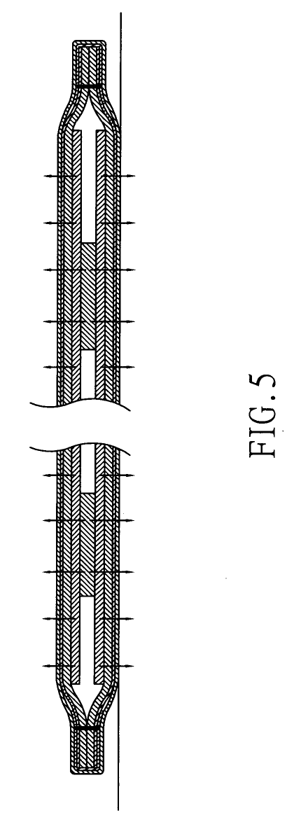

With reference to FIGS. 2 to 5, the sap adjuster of the present invention is illustrated. The sap adjuster includes a plurality of hot compress unit 10. The hot compress unit 10 includes a heat-tolerable insulting rubber sheet 12. Two electric heated sheets 14 are spaced with a predetermined distance and are adhered to the heat-tolerable insulting rubber sheet 12 by silicon. Each of the electric heated sheet 14 is connected to a long conductive wire 18. One end of one electric heated sheet 14 is connected to the one end of another electric heated sheet 14 by using a short conductive wire 16. Then, the plurality of hot compress units 10 are arranged spaced as two banks. Then the plurality of hot compress units 10 are adhered between two large area heat-tolerable insulting rubber sheets 20 by using silicon. The large area heat-tolerable insulting rubber sheet 20 is bendable. The long conductive wires 18 of the plurality of hot compress units 10 are arranged at two sides of the large area heat-tolerable insulting rubber sheet 20. Then the plurality of long conductive wires 18 are connected in parallel as two conductive wires assemblies 182. One end of each conductive wires assembly 182 is connected to a plug 184 which can be connected to a receptacle 32 of a control box 30 for hot compress.

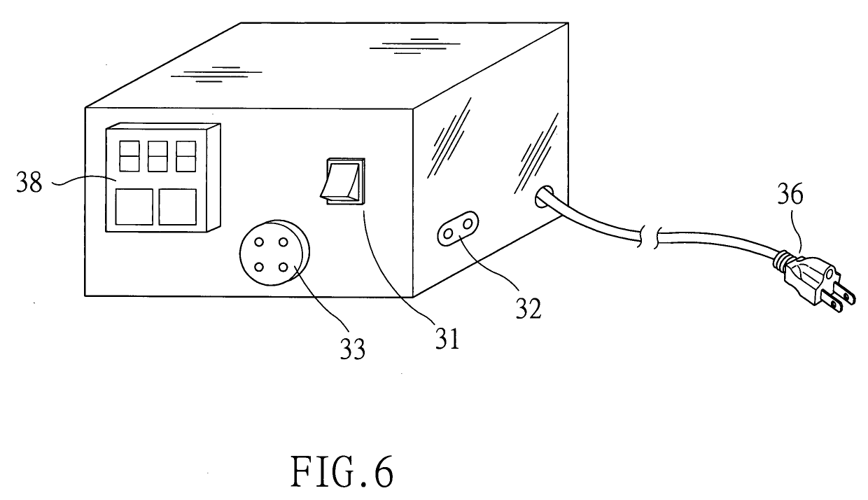

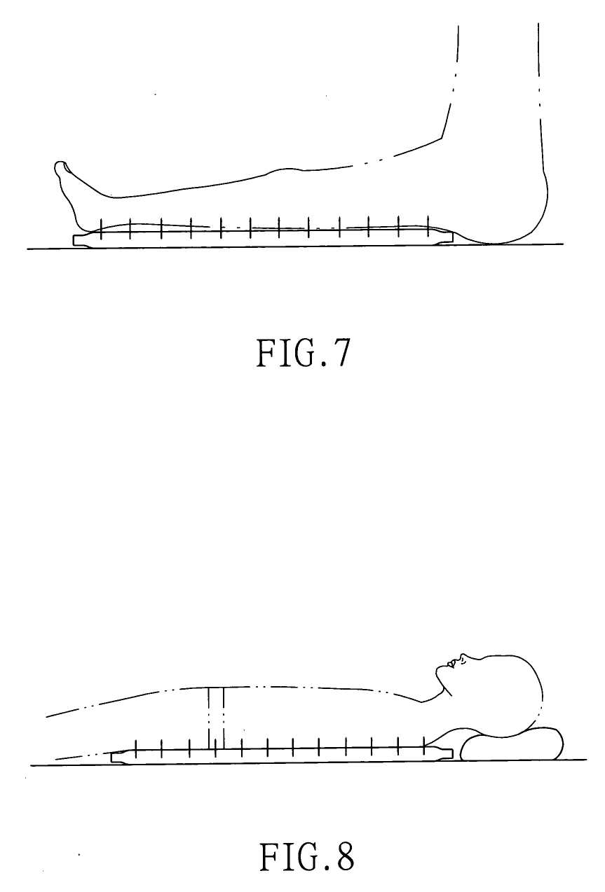

With reference to FIGS. 2 to 6, one end surface of the control box 30 is installed with a receptacle 32, and a power plug 36. Another end surface of the control box 30 is installed with a temperature controller 38, a power switch 31, and a four hole receptacle 33 for providing plugs of different types. Each unit of the temperature scale of the temperature controller 38 is two degrees so that the temperature is adjusted rapidly.

Thereby, by above said structure, the temperature of the whole hot compress unit is adjustable by adjusting the temperature controller of the control box for matching the requirement of different patients.

Thereby, in the present invention, a plurality of hot compress units 10 are connected in parallel so as to be formed as a large area heat-tolerable insulting rubber sheet 20. The long conductive wires of electric heated sheets are collected at the edges of the large area heat-tolerable insulting rubber sheet. The upper sides and lower sides of the plurality of hot compress units 10 are enclosed by two large area heat-tolerable insulting rubber sheets 20 so that the whole sap adjuster of the present invention can be bent with the shape of the human body. Thus, the present invention is a device of matching ergonomics.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A sap adjuster comprising
a plurality of hot compress units; each hot compress unit having a heat-tolerable insulting rubber sheet and two electric heated sheets; the heat-tolerable insulting rubber sheet and the two electric heated sheet being combined by silicon; a short conductive wire serving to connect the two electric heated sheets; each electric heated sheet being connected with a long conductive wire; and
a control box comprising at least one receptacle, and a power plug; a temperature controller, and a power switch.

2. The sap adjuster as claimed in claim 1, wherein the at least one receptacle contains a four hole receptacle.

3. The sap adjuster as claimed in claim 1, wherein the plurality of hot compress units are arranged in parallel so as to be as a hot compress assembly which has a larger area.

4. The sap adjuster as claimed in claim 1, wherein all the long conductive wires are arranged at two sides of the hot compress assembly.

5. The sap adjuster as claimed in claim 2, wherein the four hole receptacle is provided for connecting plugs of different types.
